(19) United States
(12) Patent Application Publication
(10) Pub. No.: US 2024/0175954 A1
(43) Pub. Date: May 30, 2024

(54) Magnetic Resonance Scanning

(71) Applicant: Siemens Healthcare GmbH, Erlangen (DE)

(72) Inventors: Jun Xiong, Shenzhen (CN); Le Zhang, Shenzhen (CN); Fang Yong Sun, Shenzhen (CN); Hui Song, Shenzhen (CN); Xu He, Shenzhen (CN)

(73) Assignee: Siemens Healthcare GmbH, Erlangen (DE)

(21) Appl. No.: 18/520,362

(22) Filed: Nov. 27, 2023

(30) Foreign Application Priority Data
Nov. 30, 2022 (CN) 202211518336.8

(51) Int. Cl. G01R 33/54; A61B 5/055

(52) U.S. Cl. CPC G01R 33/543; A61B 5/055

(57) ABSTRACT

A magnetic resonance scanning method including: detecting the positions of individual characteristic points of interest and the positions and sizes of individual sites of interest of a target human body in the current MR scan; calculating a scan start position and a total scan range of the current MR scan according to the detected positions of the individual characteristic points of interest and the positions and sizes of the individual sites of interest of the target human body; and performing the current MR scan according to the calculated scan start position and total scan range of the MR scan. In an aspect, the scanning method is fully automatic.

### TECHNICAL FIELD

The present disclosure relates to the field of Magnetic Resonance Imaging (MRI) technology, in particular to Magnetic Resonance (MR) scanning methods and devices, as well as MRI systems.

### BACKGROUND

In the clinical application of magnetic resonance imaging (MRI) scanning, to support applications such as whole-spine scanning, scanning of blood vessels of the lower extremity, and whole-body tumor scanning, most magnetic resonance imaging systems provide a multi-station scanning or Movement During Scan (MDS) method to support a wider scan range.

For the Movement During Scan mode, a scan start position and a scan range need to be set in the scanning protocol. For a cyclic multi-station scan, for example, a whole-spine scan, the number of stations generally defaults to 3, while for lower extremities CT angiography, the number of stations defaults to 9 in the Quiescent Interval Single Shot (QISS) protocol. For a single-step multi-station scan, for example, a whole-body or half-body tumor scan, separate scanning positions, and scan ranges need to be set for each station.

An obvious problem with an existing default scanning protocol is that in the scanning protocol, the number of stations to be scanned, as well as the scanning location and range, are set according to an average height only. When the patient to be scanned is a child or is relatively short or tall, an operator needs to manually adjust the scanning position according to the patient's height; otherwise, the scanning results obtained will be outside a suitable range.

FIG. 1 is a schematic diagram comparing the scanning effects on patients of different heights when a cyclic three-station scanning protocol is adopted in an existing spine scan. As shown in FIG. 1, the three-station scanning protocol for a spine scan defines a scan range of 40 centimeters (cm) for each station, with a total scan range of 82 cm, wherein the scan ranges for each station are indicated by 111-113, respectively. It is thus clear that a scan range of 82 cm defined in the protocol fits a patient with an average height, for example, the patient 12, who is 170 cm tall and is in the middle of FIG. 1, but for a short patient, for example, the patient 11, who is 150 cm tall and is on the left of FIG. 1, a two-station scan is sufficient to obtain an image of the entire spine; for a tall patient, for example, the patient 13, who is 190 cm tall and is on the right of FIG. 1, the entire spine cannot be fully covered by the scanning protocol, and manual adjustment is required.

FIG. 2 is a schematic diagram of the scanning effects on patients of different heights when the Movement During Scan protocol is adopted in an existing scan of blood vessels of a lower extremity, wherein the scanning parameters are listed in Table 1:

TABLE 1

| | |
|---|---|
| Scan start position | 24.7 cm (distance from the |
| | scanning reference line) |
| Scan range | 149.5 cm |

As clearly shown in FIG. 2, the default scan range of 149.5 cm cannot fully fit patients in all the height ranges. The scan range is indicated by 24. It is clear that for the patient 21 on the left, who is 150 cm tall, the scan range is too large, while for patient 22 in the middle, who is 170 cm tall, and the patient 23 on the right, who is 190 cm tall, the scan range is also slightly too large, which may result in a lengthy scan.

FIG. 3 is a schematic diagram of the scanning effect on patients of different heights when a cyclic multi-station scanning protocol is adopted in the scanning of blood vessels of a lower extremity. The scanning parameters are listed in Table 2:

TABLE 2

| | |
|---|---|
| Scanning position of the first station | 8.9 cm (distance from the |
| | centerline of the first station |
| | to the scanning reference line) |
| Scan range for each station | 12.06 cm |

As clearly shown in FIG. 3, the default scan range for each station, which is 12.06 cm, and the default number of stations, which is 9, cannot fully fit patients in all the height ranges. For the patient 31 on the left, who is 150 cm tall, the scan range is too large; for the patient 33 on the right, who is 190 cm tall, the scan range is slightly too small; for the patient in the middle who is 170 cm tall, the scan range is suitable.

FIG. 4 is a schematic diagram of the scanning effects on patients of different heights when a single-step multi-station scanning protocol is adopted in an existing whole-body tumor scan. For a single-step multi-station scan, the positions and scan ranges of each station need to be defined separately. The scanning durations for individual sites when a positioning image is scanned using a single-step multi-station scanning protocol are listed in Table 3:

TABLE 3

| Scanned site | Scanning duration (seconds) |
|---|---|
| Head and neck | 9 |
| Thoracic cavity | 18 |
| Abdomen | 18 |
| Pelvis | 9 |
| Thigh | 9 |
| Shank | 9 |
| Foot | 9 |

As shown in Table 3, since different sites may have different scan ranges, the scanning durations of different sites may vary.

In FIG. 4, the one-step multi-stop scanning protocol consists of 7 stations, with the scan ranges of each station indicated by 401-407, and there are overlapping areas between adjacent stations. As clearly shown in FIG. 4, the default protocol is suitable only for the patient 43, who is 190 cm tall, while for the patients 41 and 42, who are 150 cm and 170 cm tall, respectively, there is the problem of an excessively large scan range.

At present, for multi-station scanning, in order to achieve a good scanning effect, a rough scan is usually performed before formal scanning to obtain a positioning image, the positions and ranges of individual sites to be scanned are determined according to the positioning image, and then an operator, according to the positions and ranges of the individual sites to be scanned, manually adjusts the scanning positions or/and scan ranges of the individual sites to be scanned. Shortcomings of this approach are that manual adjustment cannot guarantee the optimal scanning effect, and that a rough positioning image scan needs to be performed before the formal scanning, which lengthens the entire scanning process.

### SUMMARY

In view of the above problems, in one aspect, an aspect of the present disclosure proposes an MR scanning method and device to improve the accuracy and efficiency of MR scanning; in another aspect, an MRI system to improve the accuracy and efficiency of MR scanning is proposed.

Disclosed is a magnetic resonance (MR) scanning method, comprising:
Detecting the positions of individual characteristic points of interest as well as the positions and sizes of individual sites of interest of the target human body in the current MR scan;
calculating a scan start position and a total scan range of the current MR scan according to the detected positions of individual characteristic points of interest as well as the detected positions and sizes of individual sites of interest of the target human body;
performing the current MR scan according to the calculated scan start position and total scan range of the MR scan.

Said detecting the positions of individual characteristic points of interest as well as the positions and sizes of individual sites of interest of the target human body in the current MR scan comprises:
capturing, with a camera, images of the target human body to be scanned in the current MR scan;
inputting the images of the target human body into a characteristic point and site detection model for calculation, and outputting, with the model, the positions of the individual characteristic points of interest as well as the positions and sizes of the individual sites of interest of the target human body in an image coordinate system; and
converting the positions of the individual characteristic points of interest as well as the positions and sizes of the individual sites of interest of the target human body in the image coordinate system to positions of individual characteristic points of interest as well as the positions and sizes of individual sites of interest of the target human body in an MR system coordinate system.

The characteristic point and site detection model is obtained by the steps of:
A. obtaining a training sample set, wherein a training sample is a human body image collected by a camera; obtaining true positions of individual characteristic points of interest as well as true positions and true sizes of individual sites of interest for each training sample;
B. inputting each training sample into a characteristic point and site detection neural network to be trained for calculation, and outputting, with the neural network, calculated positions of individual characteristic points of interest as well as calculated positions and calculated sizes of individual sites of interest for each training sample;
C. calculating a loss function according to the calculated positions of individual characteristic points of interest as well as the calculated positions and calculated sizes of individual sites of interest for each training sample, and the true positions of individual characteristic points of interest as well as the true positions and true sizes of individual sites of interest for each training sample;
D. adjusting the characteristic point and site detection neural network according to the loss function;
E. repeating the steps B to D until the characteristic point and site detection neural network converges, and using the converged characteristic point and site detection neural network as the characteristic point and site detection model.

Said obtaining the true positions of individual characteristic points of interest as well as the true positions and true sizes of individual sites of interest for each training sample comprises:
obtaining MR images corresponding to each training sample, the MR images being marked with the true positions of individual characteristic points of interest as well as the true positions and true sizes of individual sites of interest; and
for any MR image, converting the true positions of individual characteristic points of interest, as well as the true positions and true sizes of individual sites of interest marked on the MR image from the MR system coordinate system to the image coordinate system to obtain the true positions of individual characteristic points of interest, as well as the true positions and true sizes of individual sites of interest for the training samples corresponding to the MR image.

Said detecting the positions of individual characteristic points of interest as well as the positions and sizes of individual sites of interest of the target human body in the current MR scan comprises:
capturing, with a camera, images of the target human body to be scanned in the current MR scan;
from the images, detecting a height and a crown position of the target human body or detecting a height and a sole position of the target human body;
determining the positions of individual characteristic points of interest of the target human body in the image coordinate system according to the ratio of the distance between the individual characteristic points of interest and the crown to the height and in combination with the height and the crown position of the target human body; alternatively, determining the positions of the individual characteristic point of interest of the target human body in the image coordinate system according to a predefined ratio of the distance between the individual characteristic point of interest and the sole to the height and in combination with the height and the sole position of the target human body;
determining the positions of individual sites of interest of the target human body in the image coordinate system according to the ratio of the distance between the individual sites of interest and the crown to the height and in combination with the height and the crown position of the target human body; alternatively, determining the positions of individual sites of interest of the target human body in the image coordinate system according to a predefined ratio of the distance between the individual sites of interest and the sole to the height and in combination with the height and the sole position of the target human body;
determining the sizes of individual sites of interest of the target human body in the image coordinate system according to a predefined ratio of the sizes of the individual sites of interest to the height and in combination with the height of the target human body;
converting the positions of the individual characteristic points of interest as well as the positions and sizes of the individual sites of interest of the target human body in the image coordinate system to the positions of the individual characteristic points of interest as well as the positions and sizes of the individual sites of interest of the target human body in the MR system coordinate system.

Said calculating the scan start position and total scan range of the current MR scan according to the detected positions of the individual characteristic points of interest as well as the positions and sizes of the individual sites of interest of the target human body comprises:
obtaining the scan start position of the current MR scan according to the characteristic point of interest corresponding to the scan start point defined in a protocol adopted in the current MR scan and in combination with the detected position of the characteristic point of interest of the target human body;
obtaining a scan end position of the current MR scan according to the characteristic point of interest corresponding to the scan end point defined in the protocol adopted in the current MR scan and in combination with the detected position of the characteristic point of interest of the target human body;
determining the total scan range of the current MR scan according to the scan start position and scan end position of the current MR scan.

Said performing the current MR scan according to the calculated scan start position and total scan range of the MR scan comprises:
performing the current MR scan directly according to the calculated scan start position and total scan range of the current MR scan if a Movement During Scan protocol is adopted in the current MR scan.

Said performing the current MR scan according to the calculated scan start position and total scan range of the MR scan comprises:
determining whether an overlap area is defined between adjacent stations in the protocol adopted in the current MR scan if a cyclic multi-station scanning protocol is adopted in the current MR scan;
if yes, calculating an optimal number of scanning stations for the current MR scan according to a minimum overlap area between adjacent stations defined by the protocol, the scan range for each station defined by the protocol, and the calculated total scan range for the current MR scan, calculating the optimal size of the overlap area between adjacent stations according to the calculated total scan range of the current MR scan and the calculated optimal number of scanning stations for the current MR scan, calculating the scanning position of each station according to the calculated scan start position of the current MR scan, the calculated optimal size of the overlap area between adjacent stations, and the scan range for each station defined by the protocol, and performing the current MR scan according to the calculated scanning position of each station in the current MR scan and the scan range for each station defined by the protocol;
if no, calculating an optimal number of scanning stations for the current MR scan according to the scan range for each station defined by the protocol and the calculated total scan range for the current MR scan, calculating an optimal scan range for each station according to the calculated total scan range for the current MR scan and the calculated optimal number of scanning stations for the current MR scan, and performing the current MR scan according to the calculated scan start position of the current MR scan and the calculated optimal scan range for each station.

Said performing the current MR scan according to the calculated scan start position and total scan range of the MR scan comprises:
if a single-step multi-station scanning protocol is adopted in the current MR scan, determining the scanning positions of each station in the current MR scan according to the characteristic points of interest defined in the protocol adopted in the current MR scan corresponding to each station and in combination with the detected positions of the characteristic points of interest of the target human body; determining the scan ranges of each station in the current MR scan according to the sites of interest defined in the protocol corresponding to each station and in combination with the detected sizes of the sites of interest of the target human body.

The method, before detecting the positions of individual characteristic points of interest as well as the positions and sizes of individual sites of interest of the target human body in the current MR scan, further comprises:
determining whether the current MR scan is a whole-body scan or a half-body scan, and, if yes, performing an action of detecting the positions of the individual characteristic points of interest as well as the positions and sizes of the individual sites of interest of the target human body in the current MR scan.

Disclosed is a magnetic resonance (MR) scanning device, comprising:
A characteristic point and site detection module configured to detect the positions of individual characteristic points of interest as well as the positions and sizes of individual sites of interest of the target human body in the current MR scan;
A scanning position and range calculation module is configured to calculate the scan start position and total scan range of the current MR scan according to the detected positions of individual characteristic points of interest as well as the positions and sizes of individual sites of interest of the target human body and to perform the current MR scan according to the calculated scan start position and a total scan range of the MR scan.

Said detecting, with the characteristic point and site detection module, the positions of individual characteristic points of interest as well as the positions and sizes of individual sites of interest of the target human body in the current MR scan comprises:
capturing, with a camera, images of the target human body to be scanned in the current MR scan;
inputting the images of the target human body into a characteristic point and site detection model for calculation, and outputting, with the model, the positions of the individual characteristic points of interest as well as the positions and sizes of the individual sites of interest of the target human body in an image coordinate system; and
converting the positions of the individual characteristic points of interest as well as the positions and sizes of the individual sites of interest of the target human body in the image coordinate system to positions of individual characteristic points of interest as well as the positions and sizes of individual sites of interest of the target human body in an MR system coordinate system.

Obtaining, with the characteristic point and site detection module, a characteristic point and site detection model into which an image of the target human body is input comprises the steps of:
A. obtaining a training sample set, wherein a training sample is a human body image collected by a camera; obtaining true positions of individual characteristic points of interest as well as true positions and true sizes of individual sites of interest for each training sample;
B. inputting each training sample into a characteristic point and site detection neural network to be trained for calculation, and outputting, with the neural network, calculated positions of individual characteristic points of interest as well as calculated positions and calculated sizes of individual sites of interest for each training sample;
C. calculating a loss function according to the calculated positions of individual characteristic points of interest as well as the calculated positions and calculated sizes of individual sites of interest for each training sample, and the true positions of individual characteristic points of interest as well as the true positions and true sizes of individual sites of interest for each training sample;
D. adjusting the characteristic point and site detection neural network according to the loss function;
E. repeating the steps B to D until the characteristic point and site detection neural network converges, and using the converged characteristic point and site detection neural network as the characteristic point and site detection model.

Said obtaining, with the characteristic point and site detection module, the true positions of individual characteristic points of interest, as well as the true positions and true sizes of individual sites of interest for each training sample, comprises:
obtaining MR images corresponding to each training sample, the MR images being marked with the true positions of individual characteristic points of interest as well as the true positions and true sizes of individual sites of interest; and
for any MR image, converting the true positions of individual characteristic points of interest, as well as the true positions and true sizes of individual sites of interest marked on the MR image from the MR system coordinate system to the image coordinate system to obtain the true positions of individual characteristic points of interest, as well as the true positions and true sizes of individual sites of interest for the training samples corresponding to the MR image.

Said detecting, with the characteristic point and site detection module, the positions of individual characteristic points of interest as well as the positions and sizes of individual sites of interest of the target human body in the current MR scan comprises:
capturing, with a camera, images of the target human body to be scanned in the current MR scan;
from the images, detecting a height and a crown position of the target human body or detecting a height and a sole position of the target human body;
determining the positions of individual characteristic points of interest of the target human body in the image coordinate system according to the ratio of the distance between the individual characteristic points of interest and the crown to the height and in combination with the height and the crown position of the target human body; alternatively, determining the positions of the individual characteristic point of interest of the target human body in the image coordinate system according to a predefined ratio of the distance between the individual characteristic point of interest and the sole to the height and in combination with the height and the sole position of the target human body;
determining the positions of individual sites of interest of the target human body in the image coordinate system according to the ratio of the distance between the individual sites of interest and the crown to the height and in combination with the height and the crown position of the target human body; alternatively, determining the positions of individual sites of interest of the target human body in the image coordinate system according to a predefined ratio of the distance between the individual sites of interest and the sole to the height and in combination with the height and the sole position of the target human body;
determining the sizes of individual sites of interest of the target human body in the image coordinate system according to a predefined ratio of the sizes of the individual sites of interest to the height and in combination with the height of the target human body;
converting the positions of the individual characteristic points of interest as well as the positions and sizes of the individual sites of interest of the target human body in the image coordinate system to the positions of the individual characteristic points of interest as well as the positions and sizes of the individual sites of interest of the target human body in the MR system coordinate system.

Said calculating the scan start position and total scan range of the current MR scan with the scanning position and range calculation module comprises:
obtaining the scan start position of the current MR scan according to a characteristic point of interest corresponding to the scan start point defined in the protocol adopted in the current MR scan, and in combination with the position of the characteristic point of interest of the target human body;
obtaining the scan end position of the current MR scan according to a characteristic point of interest corresponding to the scan end point defined in the protocol adopted in the current MR scan, and in combination with the position of the characteristic point of interest of the target human body;
determining the total scan range of the current MR scan according to the scan start position and scan end position of the current MR scan.

Said performing, with the scanning position and range calculation module, the current MR scan according to the calculated scan start position and total scan range of the MR scan comprises:
performing the current MR scan directly according to the calculated scan start position and total scan range of the current MR scan if a Movement During Scan protocol is adopted in the current MR scan.

Said performing, with the scanning position and range calculation module, the current MR scan according to the calculated scan start position and total scan range of the MR scan comprises:
determining whether an overlap area is defined between adjacent stations in the protocol adopted in the current MR scan if a cyclic multi-station scanning protocol is adopted in the current MR scan;
if yes, calculating an optimal number of scanning stations for the current MR scan according to a minimum overlap area between adjacent stations defined by the protocol, the scan range for each station defined by the protocol, and the calculated total scan range for the current MR scan, calculating an optimal size of the overlap area between adjacent stations according to the calculated total scan range of the current MR scan and the calculated optimal number of scanning stations for the current MR scan, calculating the scanning position for each station according to the calculated scan start position of the current MR scan, the calculated optimal size of the overlap area between adjacent stations, and the scan range for each station defined by the protocol, and performing the current MR scan according to the calculated scanning positions of each station in the current MR scan and the scan range for each station defined by the protocol;
if no, calculating an optimal number of scanning stations for the current MR scan according to the scan range for each station defined by the protocol and the calculated total scan range for the current MR scan, calculating an optimal scan range for each station according to the calculated total scan range for the current MR scan and the calculated optimal number of scanning stations for the current MR scan, and performing the current MR scan according to the calculated scan start position of the current MR scan and the optimal scan range for each station.

Said performing, with the scanning position and range calculation module, the current MR scan according to the scan start position and scan range of the current MR scan comprises:
if a single-step multi-stop scanning protocol is adopted in the current MR scan, determining the scanning positions of each station according to the names of the characteristic points of interest defined in the protocol and the positions of the obtained characteristic points of interest, and determining the scan ranges of each station according to the names of the sites of interest corresponding to each station defined in the protocol and the sizes of the corresponding obtained sites of interest.

Disclosed is a magnetic resonance imaging (MRI) system comprising a magnetic resonance (MR) scanning device as described above.

In an aspect of the present disclosure, scanning positions and ranges are adaptively adjusted according to the positions of the characteristic points of interest as well as the positions and sizes of the sites of interest of the target human body, accurately adapting to target human bodies with various heights to be scanned, which solves the problems of inaccurate scanning positions and excessively large or small scan ranges caused by differences in target human body height and makes the scanning process fully automatic, thus improving the scanning accuracy and scanning efficiency.

### BRIEF DESCRIPTION OF THE DRAWINGS

Preferred aspects of the present disclosure are described in detail below with reference to the drawings to give those skilled in the art a clearer understanding of the above-mentioned and other features and advantages of the present disclosure. In the figures:

FIG. 1 is a schematic diagram comparing the scanning effects on patients of different heights when a cyclic three-station scanning protocol is adopted in an existing spine scan;

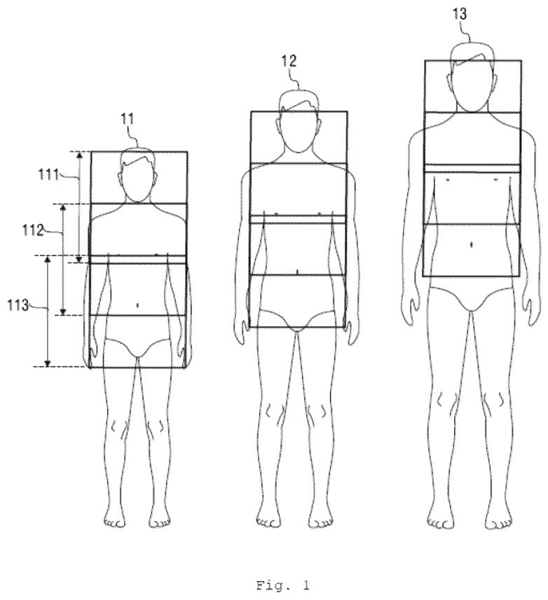

FIG. 2 is a schematic diagram of the scanning effects on patients of different heights when the Movement During Scan protocol is adopted in an existing scan of blood vessels of the lower extremity;

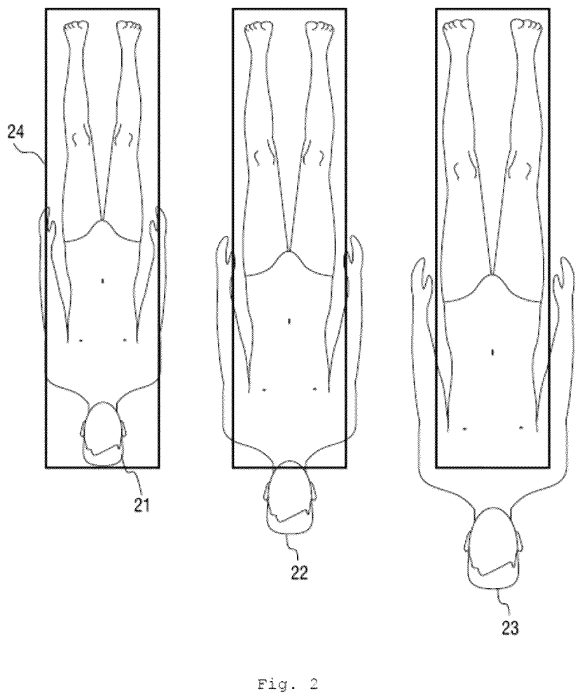

FIG. 3 is a schematic diagram of the scanning effect on patients of different heights when a cyclic multi-station scanning protocol is adopted in the scanning of blood vessels of the lower extremity;

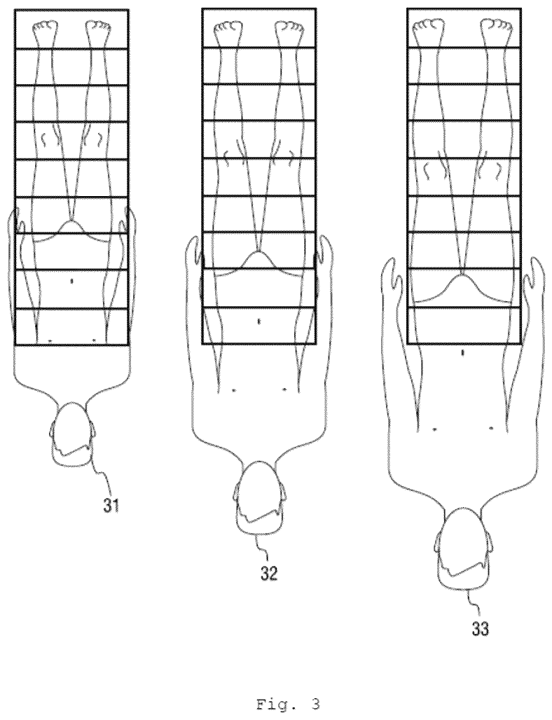

FIG. 4 is a schematic diagram of the scanning effects on patients of different heights when a single-step multi-station scanning protocol is adopted in an existing whole-body tumor scan;

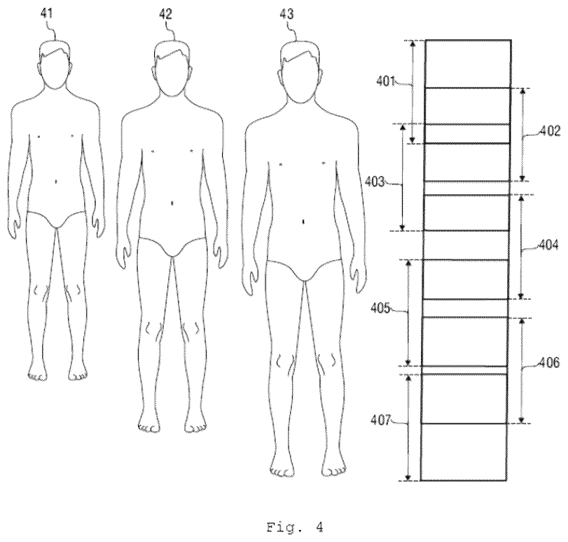

FIG. 5 is a flowchart of an MR scanning method provided in an aspect of the present disclosure;

FIG. 6 is a schematic diagram of the positions of individual characteristic points of interest as well as the positions and sizes of individual sites of interest of the target human body detected using an aspect of the present disclosure;

FIG. 7 is a schematic diagram of the positional relationship between the camera and the MR system when a camera is used to capture an image of the target human body to be scanned in the current MR scan in an aspect of the present disclosure;

FIG. 8 is a schematic diagram of the scanning effects on patients of different heights when the Movement During Scan protocol is adopted in scanning of blood vessels of the lower extremity after an aspect of the present disclosure is used;

FIG. 9 is a flowchart of a method provided by an aspect of the present disclosure for performing the current MR scan in step 503 according to the calculated scan start position and total scan range of the current MR scan when a cyclic multi-station scanning protocol is adopted for the current MR scan;

FIG. 10 is a schematic diagram comparing the scanning effects on patients of different heights after an aspect of the present disclosure is used when a cyclic multi-station scanning protocol is adopted in a spine scan;

FIG. 11 is a schematic diagram of the scanning effects on patients of different heights after an aspect of the present disclosure is used when a cyclic multi-station scanning protocol is adopted in the scanning of blood vessels of the lower extremity; and

FIG. 12 is a schematic diagram of the structure of an MR scanning device provided in an aspect of the present disclosure.

The meanings of the reference signs are as follows:

| Reference sign | Meaning |
|---|---|
| 11 | Patient with a height of 150 cm |
| 12 | Patient with a height of 170 cm |
| 13 | Patient with a height of 190 cm |
| 111-113 | Scan range for each station when a cyclic three-station |
| | scanning protocol is adopted in an existing spine scan |
| 21 | Patient with a height of 150 cm |
| 22 | Patient with a height of 170 cm |
| 23 | Patient with a height of 190 cm |
| 24 | Scan range when the Movement During Scan protocol |
| | is adopted in an existing scan of blood vessels of the |
| | lower extremity |
| 31 | Patient with a height of 150 cm |
| 32 | Patient with a height of 170 cm |
| 33 | Patient with a height of 190 cm |
| 41 | Patient with a height of 150 cm |
| 42 | Patient with a height of 170 cm |
| 43 | Patient with a height of 190 cm |
| 401-407 | Scan range for each station when a single-step multi- |
| | station scanning protocol is adopted in an existing |
| | whole-body tumor scan |
| 501-503 | Step |
| 71 | Camera |
| 72 | Magnet of the MR system |
| 73 | Examination couch |
| 74 | Support device of the camera |
| 75 | Connection member |
| 76 | Computer |
| 77 | Field of view of the camera |
| 81 | Patient with a height of 150 cm |
| 82 | Patient with a height of 170 cm |
| 83 | Patient with a height of 190 cm |
| 901-909 | Step |
| 101 | Patient with a height of 150 cm |
| 102 | Patient with a height of 170 cm |
| 103 | Patient with a height of 190 cm |
| 111 | Patient with a height of 150 cm |
| 112 | Patient with a height of 170 cm |
| 113 | Patient with a height of 190 cm |
| 120 | MR scanning device |
| 121 | Characteristic point and site detection module |
| 122 | Scanning position and range calculation module |

### DETAILED DESCRIPTION

To make clearer the objectives, technical solutions, and benefits of the present disclosure, the present disclosure will be described in greater detail below with reference to aspects.

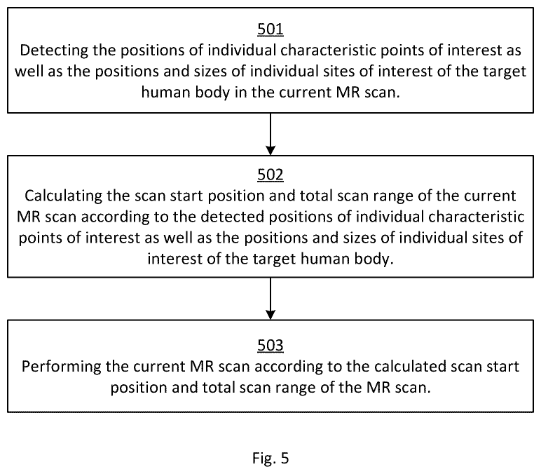

FIG. 5 is a flowchart of an MR scanning method provided in an aspect of the present disclosure, the method comprising the steps:
Step 501: Detecting the positions of individual characteristic points of interest as well as the positions and sizes of individual sites of interest of the target human body in the current MR scan.

Characteristic points of interest include the center points of human joints or anatomical sites commonly scanned in MR scanning, such as the crown, glabella, jaw, shoulder, ankle, and knee. Sites of interest include the head, thoracic cavity, abdomen, and lower extremities.

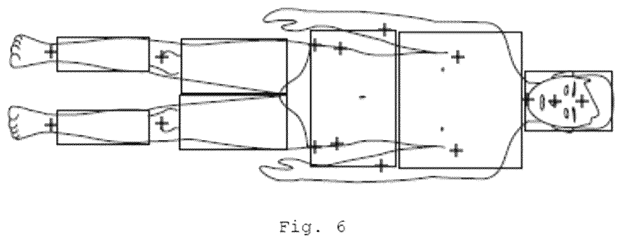

FIG. 6 is a schematic diagram of the positions of individual characteristic points of interest as well as the positions and sizes of individual sites of interest of the target human body detected using an aspect of the present disclosure. In the figure, a cross represents a characteristic point of interest, and the position of a characteristic point of interest is indicated by the position of the center point of a cross; a rectangular box represents a site of interest, and the position of a site of interest may be indicated by the position of the top left vertex or center point of a rectangular box, wherein the size of the rectangular box is the size of the site of interest.
Step 502: Calculating the scan start position and total scan range of the current MR scan according to the detected positions of individual characteristic points of interest as well as the positions and sizes of individual sites of interest of the target human body.
Step 503: Performing the current MR scan according to the calculated scan start position and total scan range of the MR scan.

In the above aspect, scanning positions and ranges are adaptively adjusted according to the positions of the characteristic points of interest as well as the positions and sizes of the sites of interest of the target human body, accurately adapting to target human bodies with various heights to be scanned, which solves the problems of inaccurate scanning positions and excessively large or small scan ranges caused by differences in target human body height and makes the scanning process fully automatic, thus improving the scanning accuracy and scanning efficiency.

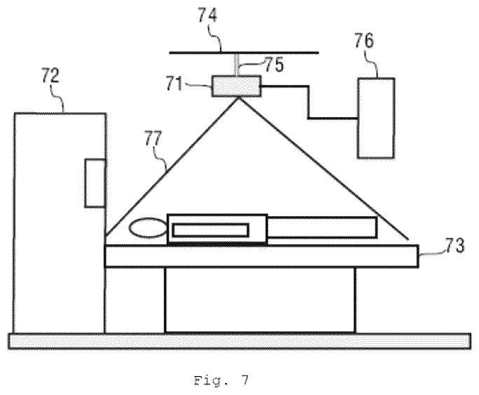

In an optional aspect, in step 501, detecting the positions of individual characteristic points of interest as well as the positions and sizes of individual sites of interest of the target human body in the current MR scan comprises:
Step 011: Capturing, with a camera, images of the target human body to be scanned in the current MR scan;
FIG. 7 is a schematic diagram of the positional relationship between the camera and the MR system when a camera is used to capture an image of the target human body to be scanned in the current MR scan in an aspect of the present disclosure. In the figure, 71 is a camera, 72 is a magnet of the MR system, 73 is an examination couch, 74 is a support device of the camera, for example, a ceiling or a magnet stand, 75 is a connection member, 76 is a computer, and 77 is the field of view of the camera, wherein the camera should be mounted in a position ensuring that the field of view of the camera covers the entire examination couch.
Step 012: From the collected image, detecting the height and the crown position of the target human body or detecting the height and the sole position of the target human body;
Step 013: Determining the positions of individual characteristic points of interest of the target human body in the image coordinate system according to the ratios of the distances between the individual characteristic points of interest and the crown to the height and in combination with the height and the crown position of the target human body; alternatively, determining the positions of the individual characteristic points of interest of the target human body in the image coordinate system according to predefined ratios of the distances between the individual characteristic points of interest and the sole to the height and in combination with the height and the sole position of the target human body;
Step 014: Determining the positions of individual sites of interest of the target human body in the image coordinate system according to the ratios of the distances between individual sites of interest and the crown to the height and in combination with the height and the crown position of the target human body; alternatively, determining the positions of individual sites of interest of the target human body in the image coordinate system according to predefined ratios of the distances between the individual sites of interest and the sole to the height and in combination with the height and the sole position of the target human body;
Step 015: Determining the sizes of individual sites of interest of the target human body in the image coordinate system according to predefined ratios of the sizes of the individual sites of interest to the height and in combination with the height of the target human body;
Herein, as attention needs to be paid only to the scanning heights of the individual sites of interest during a scan, the sizes of the individual sites of interest in this step are usually represented by the vertical heights of the individual sites of interest, namely the vertical distances between the highest points and lowest points of the individual sites of interest when the target human body is standing perpendicular to the ground.
Step 016: Converting the positions of individual characteristic points of interest as well as the positions and sizes of individual sites of interest of the target human body in the image coordinate system to the positions of individual characteristic points of interest as well as the positions and sizes of individual sites of interest of the target human body in the MR system coordinate system.

The mapping relationship between the image coordinate system and the MR system coordinate system may be measured in advance, and, according to this mapping relationship, the positions of individual characteristic points of interest as well as the positions and sizes of individual sites of interest of the target human body in the image coordinate system may be converted into the positions of the individual characteristic points of interest as well as the positions and sizes of the individual sites of interest of the target human body in the MR system coordinate system.

It should be noted that the “distances” mentioned in steps 013 and 014 described above refer to vertical distances, wherein, for example, the “distances between individual characteristic points of interest and the crown” in step 013 refer to the vertical distances from the individual characteristic points of interest to the crown when the target human body is standing perpendicular to the ground.

In an optional aspect, in step 501, detecting the positions of individual characteristic points of interest as well as the positions and sizes of individual sites of interest of the target human body in the current MR scan comprises:
Step 021: Capturing, with a camera, images of the target human body to be scanned in the current MR scan;
Images herein may be RGB images, depth maps, or RGB images and depth maps.

The positional relationship between the camera and the MR system may be as shown in FIG. 7.
Step 022: Inputting the images of the target human body into a characteristic point and site detection model for calculation and outputting, with the model, the positions of individual characteristic points of interest as well as the positions and sizes of individual sites of interest of the target human body in an image coordinate system; and
Step 023: Converting the positions of individual characteristic points of interest as well as the positions and sizes of individual sites of interest of the target human body in the image coordinate system to the positions of individual characteristic points of interest as well as the positions and sizes of individual sites of interest of the target human body in the MR system coordinate system.

In an optional aspect, the characteristic point and site detection model in step 022 is obtained by the steps:
A. obtaining a training sample set, wherein a training sample is a human body image collected by a camera; obtaining true positions of individual characteristic points of interest as well as true positions and true sizes of individual sites of interest for each training sample;
Said obtaining the true positions of individual characteristic points of interest as well as the true positions and true sizes of individual sites of interest for each training sample comprises obtaining MR images corresponding to each training sample, the MR images being marked with the true positions of individual characteristic points of interest as well as the true positions and true sizes of individual sites of interest; and for any MR image, converting the true positions of the individual characteristic points of interest, as well as the true positions and true sizes of individual sites of interest marked on the MR image from the MR system coordinate system to the image coordinate system to obtain the true positions of the individual characteristic points of interest, as well as the true positions and true sizes of the individual sites of interest for the training samples corresponding to the MR image. The MR image may be an MR positioning image or an MR clinical image.
B. inputting each training sample into a characteristic point and site detection neural network to be trained for calculation, and outputting, with the neural network, calculated positions of individual characteristic points of interest as well as calculated positions and calculated sizes of individual sites of interest for each training sample;
C. calculating a loss function according to the calculated positions of individual characteristic points of interest as well as the calculated positions and calculated sizes of individual sites of interest for each training sample, and the true positions of individual characteristic points of interest as well as the true positions and true sizes of individual sites of interest for each training sample;
D. adjusting the characteristic point and site detection neural network according to the loss function;
E. Repeating steps B to D until the characteristic point and site detection neural network have converged, and using the converged characteristic point and site detection neural network as the characteristic point and site detection model.

In an optional aspect, in step 502, calculating the scan start position and total scan range of the current MR scan according to the detected positions of individual characteristic points of interest as well as the positions and sizes of individual sites of interest of the target human body comprises:
Step 031: Obtaining the scan start position of the current MR scan according to the characteristic point of interest corresponding to the scan start point defined in the protocol adopted in the current MR scan and in combination with the detected position of the characteristic point of interest of the target human body;
Step 032: Obtaining the scan end position of the current MR scan according to the characteristic point of interest corresponding to the scan end point defined in the protocol adopted in the current MR scan and in combination with the detected position of the characteristic point of interest of the target human body;
Step 033: Determining the total scan range of the current MR scan according to the scan start position and scan end position of the current MR scan.

In an optional aspect, in step 503, performing the current MR scan according to the calculated scan start position and total scan range of the current MR scan comprises performing the current MR scan directly according to the calculated scan start position and total scan range of the current MR scan if the Movement During Scan protocol is adopted in the current MR scan.

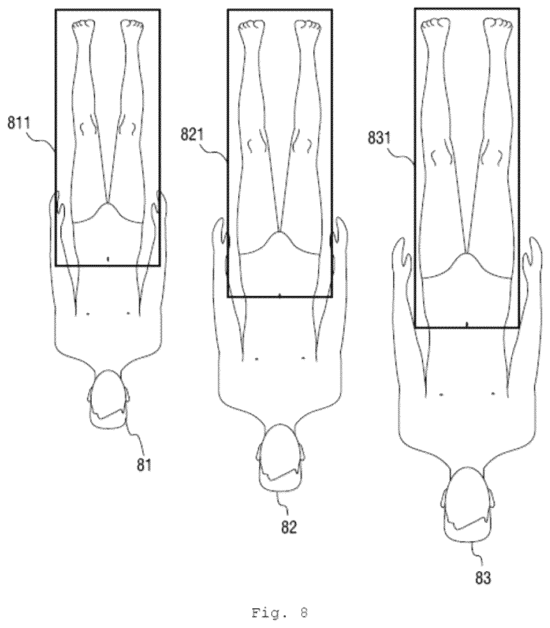

FIG. 8 is a schematic diagram of the scanning effects on patients of different heights when the Movement During Scan protocol is adopted in the scanning of blood vessels of a lower extremity after an aspect of the present disclosure is used. As shown in FIG. 8, using an aspect of the present disclosure, the scan range of patient 81 with a height of 150 cm is calculated to be 811, the scan range of patient 82 with a height of 170 cm is calculated to be 821, and the scan range of patient 83 with a height of 190 cm is calculated to be 831, wherein the scan ranges 811, 821, and 831 increase sequentially, perfectly fitting the scanned sites of each patient.

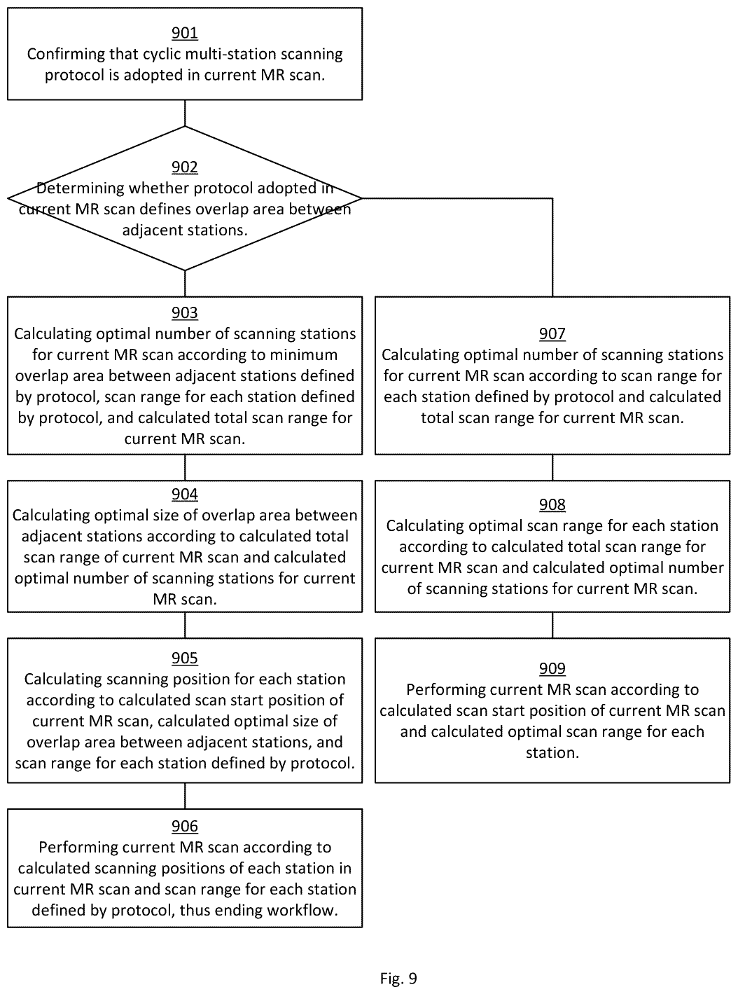

FIG. 9 is a flowchart of a method provided by an aspect of the present disclosure for performing the current MR scan in step 503 according to the calculated scan start position and total scan range of the current MR scan when a cyclic multi-station scanning protocol is adopted for the current MR scan, the method comprising the steps of:
Step 901: Confirming that a cyclic multi-station scanning protocol is adopted in the current MR scan.
Step 902: Determining whether the protocol adopted in the current MR scan defines an overlap area between adjacent stations; if yes, proceed to step 903; if no, proceed to step 907.
Step 903: Calculating the optimal number of scanning stations for the current MR scan according to the minimum overlap area between adjacent stations defined by the protocol, the scan range for each station defined by the protocol, and the calculated total scan range for the current MR scan.

For example, if the protocol defines the minimum overlap area between adjacent stations as a cm and the scan range for each station as b cm, and the total scan range of the current MR scan calculated in step 502 is n cm, then assuming the optimal number of scanning stations for the current MR scan is m, according to n=m*b−(m−1)*a, m=┌(n−a)/(b−a)┐ is calculated, wherein ┌ ┐ is the round-up operator.
Step 904: Calculating the optimal size of the overlap area between adjacent stations according to the calculated total scan range of the current MR scan and the calculated optimal number of scanning stations for the current MR scan.

For example, if the total scan range for the current MR scan calculated in step 502 is n cm, the optimal number of scanning stations for the current MR scan calculated in step 903 is m, and the scan range for each station defined in the protocol is b cm, then assuming the optimal size of the overlap area between adjacent stations is a′, according to n=m*b−(m−1)*a′, a′=(m*b−n)/(m−1) is calculated.
Step 905: Calculating the scanning position for each station according to the calculated scan start position of the current MR scan, the calculated optimal size of the overlap area between adjacent stations, and the scan range for each station defined by the protocol.
Step 906: Performing the current MR scan according to the calculated scanning positions of each station in the current MR scan and the scan range for each station defined by the protocol, thus ending the workflow.
Step 907: Calculating the optimal number of scanning stations for the current MR scan according to the scan range for each station defined by the protocol and the calculated total scan range for the current MR scan.

For example, if the protocol defines the scan range for each station as b cm and the total scan range of the current MR scan calculated in step 502 is n cm, then assuming the optimal number of scanning stations for the current MR scan is m, according to n=m*b, m=┌n/b┐ is calculated, wherein ┌ ┐ is the round-up operator.
Step 908: Calculating the optimal scan range for each station according to the calculated total scan range for the current MR scan and the calculated optimal number of scanning stations for the current MR scan.

For example, if the total scan range for the current MR scan calculated in step 502 is n cm and the optimal number of scanning stations for the current MR scan calculated in step 907 is m, then assuming the optimal scan range for each station is b′, according to n=m*b′, b′=n/m is calculated.
Step 909: Performing the current MR scan according to the calculated scan start position of the current MR scan and the calculated optimal scan range for each station.

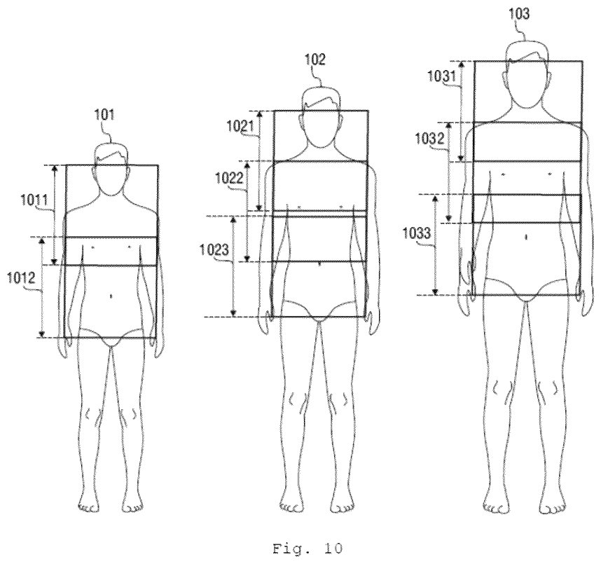

FIG. 10 is a schematic diagram comparing the scanning effects on patients of different heights after an aspect of the present disclosure is used when a cyclic multi-station scanning protocol is adopted in a spine scan, wherein, in this scanning protocol, an overlap area is defined between adjacent stations and the scan range of each station is fixed at 40 cm. As shown in FIG. 10, calculations using an aspect of the present disclosure revealed that only two stations are needed for the patient 101, with a height of 150 cm on the left side of FIG. 10, and the scan ranges of each station are indicated by 1011-1022; three stations are needed for the patient 102 with a height of 170 cm in the middle of FIG. 10, and the scan ranges of each station are indicated by 1021-1023; three stations are also needed for the patient 103 with a height of 190 cm on the right side of FIG. 10, and the scan ranges of each station are indicated by 1031-1033. To achieve the optimal scanning effect, for patients 102 and 103, the sizes of overlap areas between stations vary, wherein, for the patient 102, the overlap area between two stations is relatively large and, for the patient 103, the overlap area between two stations is relatively small. It is thus clear that after an aspect of the present disclosure is used, the scan ranges of the patients with three different heights perfectly fit the sites to be scanned. Experiments have shown that scanning with two stations takes nearly 30 seconds less than scanning with three stations.

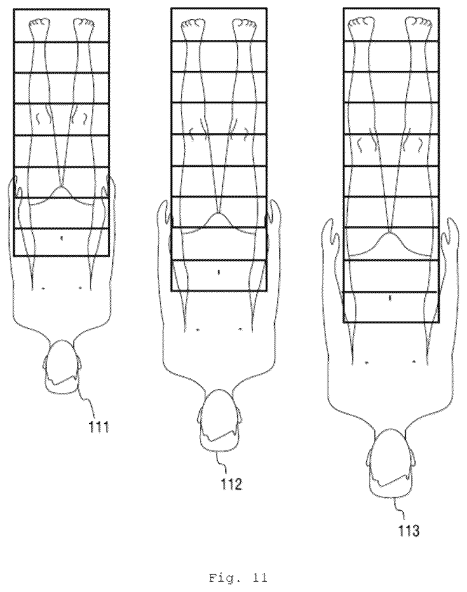

FIG. 11 is a schematic diagram of the scanning effects on patients of different heights after an aspect of the present disclosure is used when a cyclic multi-station scanning protocol is adopted in the scanning of blood vessels of a lower extremity, wherein there is no overlap area between adjacent stations. As shown in FIG. 11, after an aspect of the present disclosure is used, only 8 stations are needed for the patient 111 with a height of 150 cm on the left side of FIG. 11, 9 stations are needed for the patient 112 with a height of 170 cm in the middle of FIG. 11, and 10 stations are needed for the patient 113 with a height of 190 cm on the right side of FIG. 11. It is thus clear that after an aspect of the present disclosure is used, the scan ranges of the patients with three different heights perfectly fit the sites to be scanned.

The aspect shown in FIG. 9 is aimed at a cyclic multi-station scanning protocol in which the scan ranges for each station are the same. In practical applications, the scan ranges for each station in a single-step multi-station scanning protocol may vary, and, in view of this situation, an aspect of the present disclosure provides the following solution:

In an optional aspect, in step 503, performing the current MR scan according to the calculated scan start position and total scan range of the MR scan comprises:
if a single-step multi-station scanning protocol is adopted in the current MR scan, determining the scanning positions of each station in the current MR scan according to the characteristic points of interest defined in the protocol adopted in the current MR scan corresponding to each station and in combination with the detected positions of the characteristic points of interest of the target human body; determining the scan ranges of each station in the current MR scan according to the sites of interest defined in the protocol corresponding to each station and in combination with the detected sizes of the sites of interest of the target human body.

In an optional aspect, in step 501, the method, before detecting the positions of individual characteristic points of interest as well as the positions and sizes of individual sites of interest of the target human body in the current MR scan, further comprises determining whether the current MR scan is a whole-body scan or a half-body scan, and, if yes, performing the action of detecting the positions of the individual characteristic points of interest as well as the positions and sizes of the individual sites of interest of the target human body in the current MR scan.

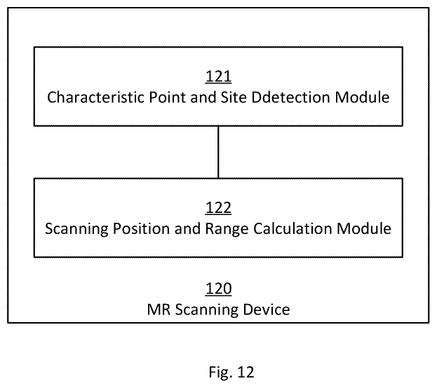

FIG. 12 is a schematic diagram of the structure of an MR scanning device 120 provided in an aspect of the present disclosure, the device comprising a characteristic point and site detection module 121 and a scanning position and range calculation module 122, wherein
a characteristic point and site detection module configured to detect the positions of individual characteristic points of interest as well as the positions and sizes of individual sites of interest of the target human body in the current MR scan; and
The scanning position and range calculation module 122 is configured to calculate the scan start position and total scan range of the current MR scan according to the positions of individual characteristic points of interest as well as the positions and sizes of individual sites of interest of the target human body detected by the characteristic point and site detection module 121; perform the current MR scan according to the calculated scan start position and total scan range of the MR scan.

In an optional aspect, detecting, with the characteristic point and site detection module 121, the positions of individual characteristic points of interest, as well as the positions and sizes of individual sites of interest of the target human body to be scanned in the current MR scan, comprises:
capturing, with a camera, images of the target human body to be scanned in the current MR scan;
From the images, detecting the height and the crown position of the target human body or detecting the height and the sole position of the target human body;
Determining the positions of individual characteristic points of interest of the target human body in the image coordinate system according to the ratios of the distances between the individual characteristic points of interest and the crown to the height and in combination with the height and the crown position of the target human body; alternatively, determining the positions of the individual characteristic points of interest of the target human body in the image coordinate system according to predefined ratios of the distances between the individual characteristic points of interest and the sole to the height and in combination with the height and the sole position of the target human body;
Determining the positions of individual sites of interest of the target human body in the image coordinate system according to the ratios of the distances between the individual sites of interest and the crown to the height and in combination with the height and the crown position of the target human body; alternatively, determining the positions of individual sites of interest of the target human body in the image coordinate system according to predefined ratios of the distances between the individual sites of interest and the sole to the height and in combination with the height and the sole position of the target human body;
Determining the sizes of individual sites of interest of the target human body in the image coordinate system according to predefined ratios of the sizes of individual sites of interest to the height and in combination with the height of the target human body;
Converting the positions of individual characteristic points of interest as well as the positions and sizes of individual sites of interest of the target human body in the image coordinate system to the positions of individual characteristic points of interest as well as the positions and sizes of individual sites of interest of the target human body in the MR system coordinate system.

In an optional aspect, detecting, with the characteristic point and site detection module 121, the positions of individual characteristic points of interest, as well as the positions and sizes of individual sites of interest of the target human body to be scanned in the current MR scan, comprises:
Capturing, with a camera, images of the target human body to be scanned in the current MR scan; inputting the images of the target human body into a characteristic point and site detection model for calculation, and outputting, with the model, the positions of individual characteristic points of interest as well as the positions and sizes of individual sites of interest of the target human body in an image coordinate system; and converting the positions of individual characteristic points of interest as well as the positions and sizes of individual sites of interest of the target human body in the image coordinate system to the positions of individual characteristic points of interest as well as the positions and sizes of individual sites of interest of the target human body in the MR system coordinate system.

In an optional aspect, obtaining, with the characteristic point and site detection module 121, a characteristic point and site detection model into which an image of the target human body is input comprises the steps of:
A. obtaining a training sample set, wherein a training sample is a human body image collected by a camera; obtaining true positions of individual characteristic points of interest as well as true positions and true sizes of individual sites of interest for each training sample;
B. inputting each training sample into a characteristic point and site detection neural network to be trained for calculation, and outputting, with the neural network, calculated positions of individual characteristic points of interest as well as calculated positions and calculated sizes of individual sites of interest for each training sample;
C. calculating a loss function according to the calculated positions of individual characteristic points of interest as well as the calculated positions and calculated sizes of individual sites of interest for each training sample, and the true positions of individual characteristic points of interest as well as the true positions and true sizes of individual sites of interest for each training sample;
D. adjusting the characteristic point and site detection neural network according to the loss function;
E. Repeating steps B to D until the characteristic point and site detection neural network has converged, and using the converged characteristic point and site detection neural network as the characteristic point and site detection model.

In an optional aspect, obtaining, with the characteristic point and site detection module 121, the true positions of individual characteristic points of interest as well as the true positions and true sizes of individual sites of interest for each training sample comprises:
Obtaining MR images corresponding to each training sample, the MR images being marked with the true positions of individual characteristic points of interest as well as the true positions and true sizes of individual sites of interest; for any MR image, converting the true positions of the individual characteristic points of interest, as well as the true positions and true sizes of the individual sites of interest marked on the MR image from the MR system coordinate system to the image coordinate system to obtain the true positions of the individual characteristic points of interest, as well as the true positions and true sizes of the individual sites of interest for the training samples corresponding to the MR image.

In an optional aspect, calculating, with the scanning position and range calculation module 122, the scan start position and total scan range of the current MR scan comprises:
Obtaining the scan start position of the current MR scan according to the characteristic point of interest corresponding to the scan start point defined in the protocol adopted in the current MR scan, and in combination with the position of the characteristic point of interest of the target human body; obtaining the scan end position of the current MR scan according to the characteristic point of interest corresponding to the scan end point defined in the protocol adopted in the current MR scan and in combination with the position of the characteristic point of interest of the target human body; determining the total scan range of the current MR scan according to the scan start position and scan end position of the current MR scan.

In an optional aspect, performing, with the scanning position and range calculation module 122, the current MR scan according to the calculated scan start position and total scan range of the current MR scan comprises:
performing the current MR scan directly according to the calculated scan start position and total scan range of the current MR scan if a Movement During Scan protocol is adopted in the current MR scan.

In an optional aspect, performing, with the scanning position and range calculation module 122, the current MR scan according to the calculated scan start position and total scan range of the current MR scan comprises:
Determining whether an overlap area is defined between adjacent stations in the protocol adopted in the current MR scan if a cyclic multi-station scanning protocol is adopted in the current MR scan; if yes, calculating the optimal number of scanning stations for the current MR scan according to the minimum overlap area between adjacent stations defined by the protocol, the scan range for each station defined by the protocol, and the calculated total scan range for the current MR scan, calculating the optimal size of the overlap area between adjacent stations according to the calculated total scan range of the current MR scan and the calculated optimal number of scanning stations for the current MR scan, calculating the scanning position for each station according to the calculated scan start position of the current MR scan, the calculated optimal size of the overlap area between adjacent stations, and the scan range for each station defined by the protocol, and performing the current MR scan according to the calculated scanning positions of each station in the current MR scan and the scan range for each station defined by the protocol; if no, calculating the optimal number of scanning stations for the current MR scan according to the scan range for each station defined by the protocol and the calculated total scan range for the current MR scan, calculating the optimal scan range for each station according to the calculated total scan range for the current MR scan and the calculated optimal number of scanning stations for the current MR scan, and performing the current MR scan according to the calculated scan start position of the current MR scan and the optimal scan range for each station.

In an optional aspect, performing, with the scanning position and range calculation module 122, the current MR scan according to the scan start position and scan range of the current MR scan comprises:
If a single-step multi-stop scanning protocol is adopted in the current MR scan, determining the scanning positions of each station according to the names of the characteristic points of interest defined in the protocol and the positions of the obtained characteristic points of interest, and determining the scan ranges of each station according to the names of the sites of interest corresponding to each station defined in the protocol and the sizes of the corresponding obtained sites of interest.

An aspect of the present disclosure further provides an MRI system comprising an MR scanning device 120 provided by an aspect of the present disclosure.

The above aspects are only preferred aspects of the present disclosure rather than being intended to limit the scope of the present disclosure, and any modifications, equivalent substitutions, improvements, etc., made without departing from the spirit or principle of the present disclosure shall fall within the scope of protection of the present disclosure.

1. A magnetic resonance (MR) scanning method, comprising:
detecting, by a characteristic point and site detection module, positions of individual characteristic points of interest and positions and sizes of individual sites of interest of a target human body in a current MR scan;
calculating, by a scanning position and range calculation module, a scan start position and a total scan range of the current MR scan according to the detected positions of individual characteristic points of interest and the detected positions and sizes of individual sites of interest of the target human body; and
performing, by the scanning position and range calculation module, the current MR scan according to the calculated scan start position and total scan range of the MR scan.

2. The method as claimed in claim 1, wherein the detecting the positions of individual characteristic points of interest and the positions and sizes of individual sites of interest of the target human body in the current MR scan comprises:
capturing, with a camera, images of the target human body to be scanned in the current MR scan;
inputting the images of the target human body into a characteristic point and site detection model for calculation, and outputting, with the model, the positions of the individual characteristic points of interest and the positions and sizes of the individual sites of interest of the target human body in an image coordinate system; and
converting the positions of the individual characteristic points of interest and the positions and sizes of the individual sites of interest of the target human body in the image coordinate system to positions of individual characteristic points of interest and the positions and sizes of individual sites of interest of the target human body in an MR system coordinate system.

3. The method as claimed in claim 2, wherein the characteristic point and site detection model is obtained by:
A. obtaining a training sample set, wherein a training sample is a human body image collected by a camera; obtaining true positions of individual characteristic points of interest and true positions and true sizes of individual sites of interest for each training sample;
B. inputting each training sample into a characteristic point and site detection neural network to be trained for calculation, and outputting, with the neural network, calculated positions of individual characteristic points of interest and calculated positions and calculated sizes of individual sites of interest for each training sample;
C. calculating a loss function according to the calculated positions of individual characteristic points of interest and the calculated positions and calculated sizes of individual sites of interest for each training sample, and the true positions of individual characteristic points of interest and the true positions and true sizes of individual sites of interest for each training sample;
D. adjusting the characteristic point and site detection neural network according to the loss function; and
E. repeating the steps B to D until the characteristic point and site detection neural network converges, and using the converged characteristic point and site detection neural network as the characteristic point and site detection model.

4. The method as claimed in claim 3, wherein the obtaining the true positions of individual characteristic points of interest and the true positions and true sizes of individual sites of interest for each training sample comprises:
obtaining MR images corresponding to each training sample, the MR images being marked with the true positions of individual characteristic points of interest and the true positions and true sizes of individual sites of interest; and
for any MR image, converting the true positions of individual characteristic points of interest, and the true positions and true sizes of individual sites of interest marked on the MR image from the MR system coordinate system to the image coordinate system to obtain the true positions of individual characteristic points of interest, and the true positions and true sizes of individual sites of interest for the training samples corresponding to the MR image.

5. The method as claimed in claim 1, wherein the detecting the positions of individual characteristic points of interest and the positions and sizes of individual sites of interest of the target human body in the current MR scan comprises:
capturing, with a camera, images of the target human body to be scanned in the current MR scan;
from the images, detecting a height and a crown position of the target human body or detecting a height and a sole position of the target human body;
determining the positions of individual characteristic points of interest of the target human body in the image coordinate system according to a ratio of a distance between the individual characteristic points of interest and the crown to the height and in combination with the height and the crown position of the target human body; alternatively, determining the positions of the individual characteristic point of interest of the target human body in the image coordinate system according to a predefined ratio of the distance between the individual characteristic point of interest and the sole to the height and in combination with the height and the sole position of the target human body;
determining the positions of individual sites of interest of the target human body in the image coordinate system according to the ratio of the distance between the individual sites of interest and the crown to the height and in combination with the height and the crown position of the target human body; alternatively, determining the positions of individual sites of interest of the target human body in the image coordinate system according to a predefined ratio of the distance between the individual sites of interest and the sole to the height and in combination with the height and the sole position of the target human body;
determining the sizes of individual sites of interest of the target human body in the image coordinate system according to a predefined ratio of the sizes of the individual sites of interest to the height and in combination with the height of the target human body; and
converting the positions of the individual characteristic points of interest and the positions and sizes of the individual sites of interest of the target human body in the image coordinate system to the positions of the individual characteristic points of interest and the positions and sizes of the individual sites of interest of the target human body in an MR system coordinate system.

6. The method as claimed in claim 1, wherein the calculating the scan start position and total scan range of the current MR scan according to the detected positions of individual characteristic points of interest and the positions and sizes of individual sites of interest of the target human body comprises:
obtaining the scan start position of the current MR scan according to the characteristic point of interest corresponding to the scan start point defined in a protocol adopted in the current MR scan and in combination with the detected position of the characteristic point of interest of the target human body;
obtaining a scan end position of the current MR scan according to the characteristic point of interest corresponding to the scan end point defined in the protocol adopted in the current MR scan and in combination with the detected position of the characteristic point of interest of the target human body; and
determining the total scan range of the current MR scan according to the scan start position and scan end position of the current MR scan.

7. The method as claimed in claim 1, wherein the performing the current MR scan according to the calculated scan start position and total scan range of the MR scan comprises:
performing the current MR scan directly according to the calculated scan start position and total scan range of the current MR scan if a Movement During Scan protocol is adopted in the current MR scan.

8. The method as claimed in claim 1, wherein the performing the current MR scan according to the calculated scan start position and total scan range of the MR scan comprises:
determining whether an overlap area is defined between adjacent stations in a protocol adopted in the current MR scan if a cyclic multi-station scanning protocol is adopted in the current MR scan;
if yes, calculating an optimal number of scanning stations for the current MR scan according to a minimum overlap area between adjacent stations defined by the protocol, the scan range for each station defined by the protocol, and the calculated total scan range for the current MR scan, calculating the optimal size of the overlap area between adjacent stations according to the calculated total scan range of the current MR scan and the calculated optimal number of scanning stations for the current MR scan, calculating a scanning position of each station according to the calculated scan start position of the current MR scan, the calculated optimal size of the overlap area between adjacent stations, and the scan range for each station defined by the protocol, and performing the current MR scan according to the calculated scanning position of each station in the current MR scan and the scan range for each station defined by the protocol; and
if no, calculating an optimal number of scanning stations for the current MR scan according to the scan range for each station defined by the protocol and the calculated total scan range for the current MR scan, calculating an optimal scan range for each station according to the calculated total scan range for the current MR scan and the calculated optimal number of scanning stations for the current MR scan, and performing the current MR scan according to the calculated scan start position of the current MR scan and the calculated optimal scan range for each station.

9. The method as claimed in claim 1, wherein the performing the current MR scan according to the calculated scan start position and total scan range of the MR scan comprises:
if a single-step multi-station scanning protocol is adopted in the current MR scan, determining the scanning positions of each station in the current MR scan according to the characteristic points of interest defined in the protocol adopted in the current MR scan corresponding to each station and in combination with the detected positions of the characteristic points of interest of the target human body; and
determining the scan ranges of each station in the current MR scan according to the sites of interest defined in the protocol corresponding to each station and in combination with the detected sizes of the sites of interest of the target human body.

10. The method as claimed in claim 1, wherein the method, before detecting the positions of individual characteristic points of interest and the positions and sizes of individual sites of interest of the target human body in the current MR scan, further comprises:
determining whether the current MR scan is a whole-body scan or a half-body scan, and, if yes, performing an action of detecting the positions of the individual characteristic points of interest and the positions and sizes of the individual sites of interest of the target human body in the current MR scan.

11. A magnetic resonance (MR) scanning device, comprising:
a characteristic point and site detection module configured to detect positions of individual characteristic points of interest and positions and sizes of individual sites of interest of a target human body in a current MR scan; and
a scanning position and range calculation module configured to: calculate a scan start position and a total scan range of the current MR scan according to the detected positions of individual characteristic points of interest and the positions and sizes of individual sites of interest of the target human body; and perform the current MR scan according to the calculated scan start position and the total scan range of the MR scan.

12. The device as claimed in claim 11, wherein the detecting, with the characteristic point and site detection module, the positions of individual characteristic points of interest and the positions and sizes of individual sites of interest of the target human body in the current MR scan comprises:
capturing, with a camera, images of the target human body to be scanned in the current MR scan;
inputting the images of the target human body into a characteristic point and site detection model for calculation, and outputting, with the model, the positions of the individual characteristic points of interest and the positions and sizes of the individual sites of interest of the target human body in an image coordinate system; and
converting the positions of the individual characteristic points of interest and the positions and sizes of the individual sites of interest of the target human body in the image coordinate system to positions of individual characteristic points of interest and the positions and sizes of individual sites of interest of the target human body in an MR system coordinate system.

13. The device as claimed in claim 11, wherein obtaining, with the characteristic point and site detection module, a characteristic point and site detection model into which an image of the target human body is input comprises steps of:
A. obtaining a training sample set, wherein a training sample is a human body image collected by a camera; obtaining true positions of individual characteristic points of interest and true positions and true sizes of individual sites of interest for each training sample;
B. inputting each training sample into a characteristic point and site detection neural network to be trained for calculation, and outputting, with the neural network, calculated positions of individual characteristic points of interest and calculated positions and calculated sizes of individual sites of interest for each training sample;
C. calculating a loss function according to the calculated positions of individual characteristic points of interest and the calculated positions and calculated sizes of individual sites of interest for each training sample, and the true positions of individual characteristic points of interest and the true positions and true sizes of individual sites of interest for each training sample;
D. adjusting the characteristic point and site detection neural network according to the loss function; and
E. repeating the steps B to D until the characteristic point and site detection neural network converges, and using the converged characteristic point and site detection neural network as the characteristic point and site detection model.

14. The device as claimed in claim 13, wherein the obtaining, with the characteristic point and site detection module, true positions of individual characteristic points of interest and true positions and true sizes of individual sites of interest for each training sample comprises:
obtaining MR images corresponding to each training sample, the MR images being marked with the true positions of individual characteristic points of interest and the true positions and true sizes of individual sites of interest; and
for any MR image, converting the true positions of individual characteristic points of interest, and the true positions and true sizes of individual sites of interest marked on the MR image from an MR system coordinate system to the image coordinate system to obtain the true positions of individual characteristic points of interest, and the true positions and true sizes of individual sites of interest for the training samples corresponding to the MR image.

15. The device as claimed in claim 11, wherein the detecting, with the characteristic point and site detection module, the positions of individual characteristic points of interest and the positions and sizes of individual sites of interest of the target human body in the current MR scan comprises:
capturing, with a camera, images of the target human body to be scanned in the current MR scan;
from the images, detecting a height and a crown position of the target human body or detecting a height and a sole position of the target human body;
determining the positions of individual characteristic points of interest of the target human body in the image coordinate system according to a ratio of a distance between the individual characteristic points of interest and the crown to the height and in combination with the height and the crown position of the target human body; alternatively, determining the positions of the individual characteristic point of interest of the target human body in the image coordinate system according to a predefined ratio of the distance between the individual characteristic point of interest and the sole to the height and in combination with the height and the sole position of the target human body;
determining the positions of individual sites of interest of the target human body in the image coordinate system according to the ratio of the distance between the individual sites of interest and the crown to the height and in combination with the height and the crown position of the target human body; alternatively, determining the positions of individual sites of interest of the target human body in the image coordinate system according to a predefined ratio of the distance between the individual sites of interest and the sole to the height and in combination with the height and the sole position of the target human body;
determining the sizes of individual sites of interest of the target human body in the image coordinate system according to a predefined ratio of the sizes of the individual sites of interest to the height and in combination with the height of the target human body; and
converting the positions of the individual characteristic points of interest and the positions and sizes of the individual sites of interest of the target human body in the image coordinate system to the positions of the individual characteristic points of interest and the positions and sizes of the individual sites of interest of the target human body in an MR system coordinate system.

16. The device as claimed in claim 11, wherein the calculating the scan start position and total scan range of the current MR scan with the scanning position and range calculation module comprises:
obtaining the scan start position of the current MR scan according to a characteristic point of interest corresponding to the scan start point defined in a protocol adopted in the current MR scan, and in combination with the position of the characteristic point of interest of the target human body;
obtaining a scan end position of the current MR scan according to a characteristic point of interest corresponding to a scan end point defined in the protocol adopted in the current MR scan, and in combination with the position of the characteristic point of interest of the target human body; and
determining the total scan range of the current MR scan according to the scan start position and scan end position of the current MR scan.

17. The device as claimed in claim 11, wherein performing, with the scanning position and range calculation module, the current MR scan according to the calculated scan start position and total scan range of the MR scan comprises:
performing the current MR scan directly according to the calculated scan start position and total scan range of the current MR scan if a Movement During Scan protocol is adopted in the current MR scan.

18. The device as claimed in claim 11, wherein performing, with the scanning position and range calculation module, the current MR scan according to the calculated scan start position and total scan range of the MR scan comprises:
determining whether an overlap area is defined between adjacent stations in a protocol adopted in the current MR scan if a cyclic multi-station scanning protocol is adopted in the current MR scan;
if yes, calculating an optimal number of scanning stations for the current MR scan according to a minimum overlap area between adjacent stations defined by the protocol, the scan range for each station defined by the protocol, and the calculated total scan range for the current MR scan, calculating an optimal size of the overlap area between adjacent stations according to the calculated total scan range of the current MR scan and the calculated optimal number of scanning stations for the current MR scan, calculating the scanning position for each station according to the calculated scan start position of the current MR scan, the calculated optimal size of the overlap area between adjacent stations, and the scan range for each station defined by the protocol, and performing the current MR scan according to the calculated scanning positions of each station in the current MR scan and the scan range for each station defined by the protocol; and
if no, calculating an optimal number of scanning stations for the current MR scan according to the scan range for each station defined by the protocol and the calculated total scan range for the current MR scan, calculating an optimal scan range for each station according to the calculated total scan range for the current MR scan and the calculated optimal number of scanning stations for the current MR scan, and performing the current MR scan according to the calculated scan start position of the current MR scan and the optimal scan range for each station.

19. The device as claimed in claim 11, wherein the performing, with the scanning position and range calculation module, the current MR scan according to the scan start position and a scan range of the current MR scan comprises:
if a single-step multi-stop scanning protocol is adopted in the current MR scan, determining the scanning positions of each station according to names of the characteristic points of interest defined in the protocol and the positions of the individual characteristic points of interest, and determining the scan ranges of each station according to names of the sites of interest corresponding to each station defined in the protocol and the sizes of the corresponding individual sites of interest.

20. A magnetic resonance imaging (MRI) system, wherein the MRI system comprises a magnetic resonance (MR) scanning device as claimed in claim 11.
